(19) United States
(12) Patent Application Publication
(10) Pub. No.: US 2011/0195493 A1
(43) Pub. Date: Aug. 11, 2011

(54) CONTINUOUS SYSTEM FOR CONVERTING CO2 INTO HIGH VALUE-ADDED AND/OR NUTRITIONAL VALUE PRODUCTS AND ENERGY RESOURCES

(75) Inventor: Bernard A.J. Stroiazzo-Mougin, Alicante (ES)

(73) Assignee: Bio Fuel Systems, S.L., Alicante (ES)

(21) Appl. No.: 12/999,890

(22) PCT Filed: Jun. 18, 2009
(86) PCT No.: PCT/ES2009/070235

§ 371 Date: Mar. 15, 2011

(30) Foreign Application Priority Data
Jun. 20, 2008 (EP) 08380179.5

(51) Int. Cl. C12M 1/00

(52) U.S. Cl. 435/292.1

(57) ABSTRACT

A system for generating high value-added or nutritional value products and other energy resources comprising first production means for culturing at least one phytoplankton culture and means for naturally and/or artificially illuminating the phytoplankton culture to induce growth and/or multiplication of the culture, wherein the systems allows for illuminating the phytoplankton culture by either external light sources or internal light sources which are independent of the external ones.

### OBJECT OF THE INVENTION

The object of the present invention is a continuous system for converting CO₂ into high value-added and/or high nutritional value products and other energy resources.

### BACKGROUND OF THE INVENTION

A huge amount of solar energy irradiates the surface of the earth, approximately about 15,000 times the current energy consumption. It is the largest energy source of our planet.

Nevertheless, the effectiveness of the concentration of energy per unit area at any point of the earth is very low, only about 1 to 5 kWh/m²/day. This low effectiveness of energy concentration limits the direct use of solar energy as a primary energy source.

An objective is to pass from this low energy density state to a high density state through a system for collecting sunlight and CO₂ to convert it into a continuous energy source using a magneto-hydro-photosynthetic catalyst, the latter being the actual system.

Based on this principle, application WO2007144440 is known which describes a photoconverter for a function similar to the present system, nevertheless, its horizontal and not vertical constitution, as well as other features, make it essentially different.

Global warming is currently a problem that is giving rise to the development of new technologies in different fields of the art in order to attempt eradicating or minimize the effects of said warming. The obligation of the economic areas to comply with the objectives imposed by the Kyoto protocol on the reduction of CO₂/SO₂ emissions and the emissions of other gases causing the so-called greenhouse effect is leading countries to search for alternative and renewable fuels to avoid possible tax penalties.

Although the production of solar and wind energy is increasing in some regions, these technologies are very expensive, they are not viable in all climatic areas and do not assure a continuous energy production. In these conditions, biofuels are intended to play an essential role as substitutes for fossil fuels, especially for transport and heating applications.

The production costs of biofuels from terrestrial plants, such as palm and rapeseed oil, have always been a reason for concern. Taking into account the low oil production indices per hectare, huge amounts of resources would be needed for a commercial production to be reached. Land and water are two scarce resources and it is preferable to use them for producing food, which are furthermore more profitable for farmers. Furthermore, intensive fertilization is a form of land and water pollution of the first order. Extensive single crop farming is also one of the main enemies of biodiversity.

A study of the University of California-Berkeley, Natural Resources Research Vol 14 No. 1 Mar. 2005 pg. 65-72 shows that a terrestrial plant, such as sunflower, requires more energy for its transformation into a fuel than the energy capable of supplying as such, for example, for the production of 1,000 kg of sunflower fuel having a calorific value of 9,000,000 Kcal, 19 million Kcal have to be consumed, which corresponds to a CO₂ emission greater than that emitted by a fossil fuel. In other words, if the emission of a 135 hp (100 kW) car on a 100 km trip making use of a fossil fuel is 20 Kg of CO₂, when a sunflower-based fuel is used, the total combined emission would be 36 Kg of CO₂. However, in the case of a fuel based on phytoplankton fed with the CO₂ of a power station, for example a thermal power station, the amount of CO₂ emitted to the atmosphere is 10 kg. This is due to the fact that the CO₂ captured by the phytoplankton has generated in turn a power of 100 kW, therefore the balance of CO₂ emitted per kW produced is half. In addition, the present invention describes a system in which not all the end product is intended for the production of fuels, but also for the production of a high nutritional value product.

It should be emphasized that when the application of the end product is that of generating electric energy, there is no CO₂ emission to the atmosphere as such CO₂ is introduced again in the system for the production of phytoplankton.

In view of that discussed above, with a production of phytoplankton which allows substituting the use of fuels of a petroliferous origin, the advantages from the environmental point of view are evident. According to this, the great challenge lies in achieving a culture which allows massively producing this product. To that end, it is essential to suitably select the reactor to be used.

The reactors that are currently used, either on an industrial or experimental scale, can be classified into two large groups:
Open reactors
Closed reactors
   Horizontal
   Vertical

### Open Reactors

They consist of the use of shallow tanks in which algae, water and nutrients circulate through the circuit by means of a motor-driven system of blades.

The great drawback represented by this system is that, since it is completely open, the algae are susceptible of being contaminated by any organism that is introduced into the tank. Furthermore, the conditions of the system, such as temperature, evaporation of water from the culture medium, supply of CO₂ and luminosity are more difficult to control, the photochemical efficiency being low (high cell densities are not reached); therefore the depth of the tank is of a few millimeters, thus requiring large areas to reach high productions. The group of these factors makes this system not be the most suitable one for the mass cultivation of phytoplankton species.

### Closed Reactors

These reactors allow overcoming some of the main obstacles faced by an open system; they allow controlling the variables of the system (CO₂, luminosity, temperature . . . ), there is no contamination, and they require smaller areas of land to reach the same production as in open reactors, especially in the event that the arrangement is vertical (greater volume per unit area). All of this makes the photochemical efficiency of the culture higher.

Due to that mentioned above, different authors have developed systems and processes in which photosynthetic phytoplankton species are cultivated which are exposed to light to generate growth and multiplication thereof in order to finally obtain a usable product (after several modification steps), as is the case of PCT applications WO2007025145A2, WO2006/020177, WO 03/094598 and WO2007144441. However all these systems have the problem that they do not work in a clearly continuous manner, do not have a suitable photochemical efficiency, do not have a recirculation of part of their own obtained products, are laborious systems which in some cases are susceptible to contamination, use solar energy in a two-dimensional manner (they do not take advantage of the verticality of the system of the present invention), and in short, have high associated costs (cleaning, disinfection, evaporation of culture water, are energetically deficient [pumping, extraction . . . ] . . . ), and which indirectly generate contamination. Furthermore, systems or processes such as that described in WO2007025145A2 work according to a horizontal arrangement (they use stirring by means of rollers), the resulting products are not reused (as is done in the present invention with the NOx passing through biofilters to be transformed into nitrates and nitrites which are used as a supply of nutrients to the culture medium). These systems or processes obtain amounts of fuels (about 6000 gallons) lower than the system of the present invention.

In addition, the system described herein has the additional advantage that in the production step a magnetic field is applied which causes an acceleration of said step and, therefore, a substantial increase of the amount of obtained product.

In this same sense, the present invention relates to a novel system for obtaining energy resources by means of a system for collecting sunlight and CO₂ for the reconversion thereof into a continuous energy source using catalysts. To minimize direct or indirect contamination, the system, which will be described below, uses nutrients such as atmospheric CO₂, carbon and nitrogen sources from different industrial sectors. By means of the present invention a total or partial recycling (depending on the application) of the CO₂ is achieved and therefore the net emission can be “zero”, since the CO₂ generated can return to the system, thus nourishing the phytoplankton. Furthermore, for the purpose of minimizing water consumption, the water is reused after the separation step which will be described in more detail below.

Furthermore, the system has the advantage that the oxygen generated in one of its steps is reused as a raw material for a subsequent transformation of the obtained product.

Another advantage is that up until now there is no system in which a mass culture of photosynthetic strains is carried out as is done in the present invention.

Thus a substantial improvement of the systems that are already known in the state of the art is achieved, either in efficiencies (energetic-economic and productive efficiencies) or in minimization of environmental impact. It is therefore a sustainable system both from the energetic and ecological point of view.

### DESCRIPTION OF THE INVENTION

To palliate the problems mentioned above, the object of the present invention is a system for generating added-value and energy resources.

Accordingly, the system for converting CO₂ into high value-added and/or high nutritional value products and other energy resources of this invention comprises:
(i) first production means (20) for culturing at least one phytoplankton culture (10), and
(ii) means for naturally and/or artificially illuminating the phytoplankton culture to induce growth and/or multiplication of the culture,
   characterized in that the said first means (20) comprise at least one tube assembly (210) which contains the phytoplankton culture (10) and which in turn comprises:
(a) a first outer tube (2101) that contains the phytoplankton culture (10) and allows exposure of the phytoplankton culture to external light sources, and
(b) a second tube (2102) inside first tube (2101) that allows exposure of the phytoplankton culture (10) to an internal light source that is independent of the external light source.

This avoids the “shading effect” that may be encountered in the inside portions of large phytoplankton culture tubes like those in the present invention, maximizing system efficiency by maximizing exposure of the phytoplankton culture to light at all points within the tube, thereby also maximizing photosynthesis by the culture.

In a preferred embodiment of the invention, the said system comprises:

(i) first production means for culturing a phytoplankton culture, wherein said first means in turn comprise:
(a) a plurality of production modules for producing phytoplankton culture that are vertically placed, supplied with a source of carbon, for instance, CO₂, and interconnected to one another in the form of a closed circuit comprising a recirculation pump for recirculating the phytoplankton culture and a plurality of interconnected production columns, and optionally means for generating a magnetic field located in the recirculation pipes, such that either a continuous or alternating excitation magnetic field is generated in said area;
(b) a structure for containing the production modules, such that preferably the module assembly structure is stackable and has dimensions such that its transport in a standard transport container is feasible;
(c) natural (sunlight, sunlight concentrators-distributors) and/or artificial (fluorescent tubes, fluorescent lamps, incandescent lamps, halogen lamps, LEDs, and radioactive light sources) means of illumination, wherein said means of illumination can be internal or external with regard to the culture production modules;
(d) optionally, means for concentrating solar radiation.

The radioactive means of generating artificial light comprise a range of means that includes nanospheres, which in turn comprise the following components:
at least one radioactive isotope in solid, liquid, or gaseous form, preferably a beta emitter; and
at least one component or compound that can be excited by the radioactive isotope and upon excitation-deexcitation emits light, preferably selected from among elements that have an unpaired electron in their last orbital.

Working in parallel is thus achieved between the production modules, assuring the same amount of fluid in a margin comprised between 10% and 15% of flow variation and one and the same load for all the modules. The system also allows an amount of processing of the phytoplankton culture of up to 20000 liters/hour, preferably 5000 liters/hour.

The production modules for producing phytoplankton culture comprise a plurality of columns connected to one other, and wherein said columns in turn comprise:

(i) a tube assembly with a maximum height of 10 meters and a preferred height between 2 and 2.25 meters, containing the phytoplankton culture and said tube assembly comprising:
(a) a first external tube comprising, in turn, at least one of the following layers:
   a first outer layer of ultraviolet treatment;
   a second intermediate layer of a material such that it is resistant to the pressure of the volume contained; and
   a third antiadherent inner layer, in contact with the contained fluid;
(b) a second internal tube with regard to the first tube, said second tube comprising at least one of the following layers:
   a first antiadherent outer layer, in contact with the contained fluid;
   a second intermediate layer of a material resistant to the pressure of the volume contained in the first tube; and
   a third inner layer of ultraviolet treatment;
(c) upper and lower assembly closure elements; and
(d) inter-column connection elements, comprising a fluid connector between columns and a plurality of auxiliary fluid circulation elements;
(e) cleaning means that comprise one or preferably two concentric rings, namely, a first outer ring and a second inner ring and wherein both are joined by means of a plurality of radii, and a peripheral element which cleans by friction the walls with which it comes into contact without damaging them;
(f) a cooling and/or refracting element (a third outer tube with regard to the first external tube which would act as a jacket), comprising in turn refracting subelements and/or a cooling and/or refracting liquid therein;
(g) a medium containing zeolites;

such that the second internal tube is inserted in the first external tube, preferably concentrically, with the assembly being closed at its ends, the inter-column connection elements being located both at its upper part and at its lower part; and

(ii) feed and turbulence generation means for generating turbulences of a fluid in gaseous state and for the feeding thereof, this fluid in gaseous state being at least one selected from:
(a) air;
(b) N₂;
(c) CO₂;
(d) industrial emission exhaust gases; or
(e) a combination thereof.

The production modules for culturing phytoplankton comprise, preferably, two to twelve, and more preferably, four production columns for culturing phytoplankton connected in parallel and/or in series and or radially. The verticality of the production modules provides clear advantages compared to the traditional horizontal placement described in the state of the art, thus, for example, the capacity of discharging the oxygen which is generated in the culture is considerably improved. Other inherent advantages are: a considerable improvement of the production, since the ratio of the volume per unit area increases; the necessary pumping requires a lower electric consumption, it being possible to stop the recirculation pump to prevent unnecessary energy consumptions during certain periods of the production step.

The modularity of the system is another considerable advantage, especially in actions of preventive maintenance and/or contamination of the phytoplankton culture, wherein only is necessary to treat the affected module and the rest of the system is not affected.

(ii) The system likewise comprises second mechanical and/or thermal separation means connected to the outlet of the first production means, such that the water present in the latter is eliminated; and wherein said separation means comprise at least one of the following:
(a) phytoplankton culture pre-concentration means, connected to the outlet of the first production means;
(b) pre-concentrated product concentration means, connected to the outlet of the pre-concentration means; and
(c) thermal drying means, connected to the outlet of the concentration means;

Among these separation means can be found, without limitation, the following means:
filtration systems;
flocculation and/or coagulation means;
ultrasonic systems;
mechanical vapor recompression (MVR) means;
centrifuging means;
pressing means;
thermal drying means using hot air and/or hot exhaust gases;
freeze-drying means;
multiple-effect evaporators;
decanting means; and
any combination of the above,

such that up to 99.9% of the water present in the culture can be removed.

(iii) The system further comprises third transformation means for transforming the dry product obtained in the second means, such that an energy resource and/or high nutritional value product is obtained, wherein said third means comprise means selected from:
(a) gasification means, such that a gas suitable for its use as a fuel is obtained; or
(b) thermochemical means of transformation, operating at all pressure ranges, e.g., by means of pyrolysis, such that a product is obtained selected from:
   a fuel oil;
   a liquid mixture of organic compounds which, after a refining process, allows obtaining different fractions with physicochemical characteristics similar to those obtained from petroleum.
   an ester which, in combination with fatty acid dissolution and extraction means and esterification/transesterification means, allows obtaining a diesel compound;
(c) nutritional product and/or value-added product extraction means;
(d) a combination of the above.

The pre-concentration means of the second separation means comprise at least one of the following systems or means:
a submerged membrane filtration system (MBR);
a tangential filtration system;
flocculation and/or coagulation means;
electrocoagulation means;
an ultrasound system;
mechanical vapor recompression (MVR) means;
centrifugation means;
decantation means; or
a combination of the above;
   such that up to 99% of the water present in the production means is eliminated.

The concentration means comprise at least one system selected from:
a mechanical vapor recompression (MVR) system;
pre-concentrated element decantation means;
centrifugation means;
pressing means; or
a combination of the above;
   such that a concentrate with a relative humidity comprised between 40% and 90%, preferably between 65 and 70%, is obtained

The drying means comprise, at least one of the following:

(i) thermal drying means for drying by hot air, such that the concentrated product can be dried by means of the insertion of hot air with a temperature not less than 75°;

(ii) lyophilization means;

(iii) multiple effect evaporators.

The invention thus described, including the vertical arrangement of the production modules, allows obtaining the following advantages:
a) containing more volume of culture per unit area, therefore more productivity per unit area;
b) that the means for generating turbulence have an effect of cleaning by friction, both for the inner wall of the outer tube and for the outer wall of the inner tube;
c) an elimination of the oxygen generated during the production step;
d) increasing the three-dimensional collection of light;
e) elimination of the shading effect
f) lower electric consumption
g) exhaust gas assimilation capacity

In a most preferred embodiment of the invention, the system is operated continuously. Nevertheless, discontinuous operation would also be feasible, though in this latter case the benefits of the invention would not be as pronounced.

The dependent claims set out herein describe additional embodiments of the invention.

### BRIEF DESCRIPTION OF THE DRAWINGS

A series of drawings is very briefly described below which aid in better understanding the invention and which are expressly related to an embodiment of said invention which is set forth as a non-limiting example thereof.

FIG. 1 shows a block diagram of the continuous system for the generation of high nutritional value and/or high value-added and/or energy resources, object of the present invention.

FIG. 2 is a top view of a system for converting CO₂ into high nutritional value and/or high value-added and/or energy resources, comprising means for culturing phytoplankton, an integral part of the system object of the present invention.

FIG. 3 shows a detailed view of the joining of the production modules for producing phytoplankton culture, an integral part of the system object of the present invention.

FIG. 4 shows a detailed view of a production module.

FIG. 5 shows an exploded view of the production module shown in FIG. 4.

FIG. 6 is a top view of phytoplankton culture production means in an arrangement of eight production modules.

FIG. 7 is a top view of a hexagonal arrangement of a system for converting CO₂ into high nutritional value and/or high value-added and/or energy resources, composed of six phytoplankton culture production means, each in turn consisting of a group of eight phytoplankton culture production modules.

FIG. 8 is a top view of a pyramid-shaped arrangement of a system for converting CO₂ into high nutritional value and/or high value-added and/or energy resources, composed of two phytoplankton culture production means, each in turn consisting of a group of 23 phytoplankton culture production modules.

FIG. 9 is a top view of a system for converting CO₂ into high nutritional value and/or high value-added and/or energy resources, composed of two phytoplankton culture production means, each in turn consisting of a group of nine phytoplankton culture production modules.

FIG. 10 plots absorbance at 446 nm on biomass (g dry matter/L) for the experiments performed.

FIG. 11 depicts a detail of the inner column in experiment N9.

FIG. 12 depicts the photobioreactors in experiments N1 and N2.

FIG. 13 charts biomass production in the various photobioreactors at the end of each experiment.

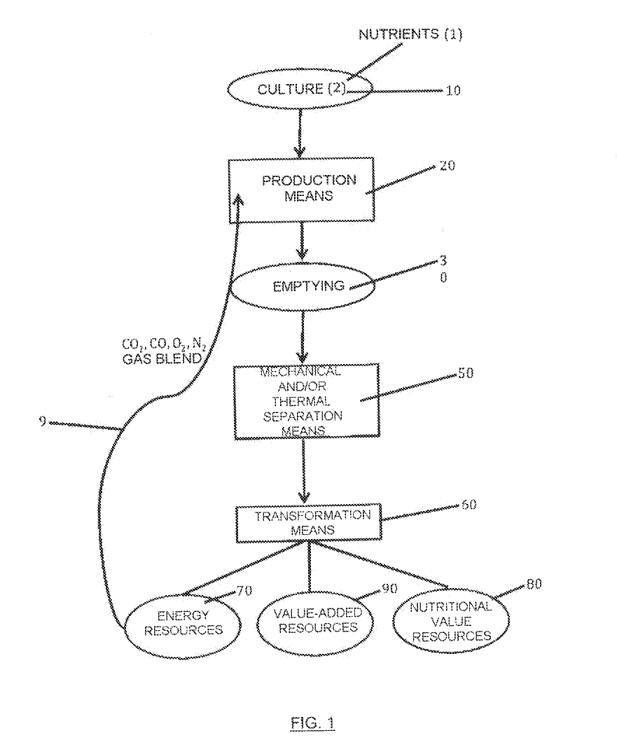

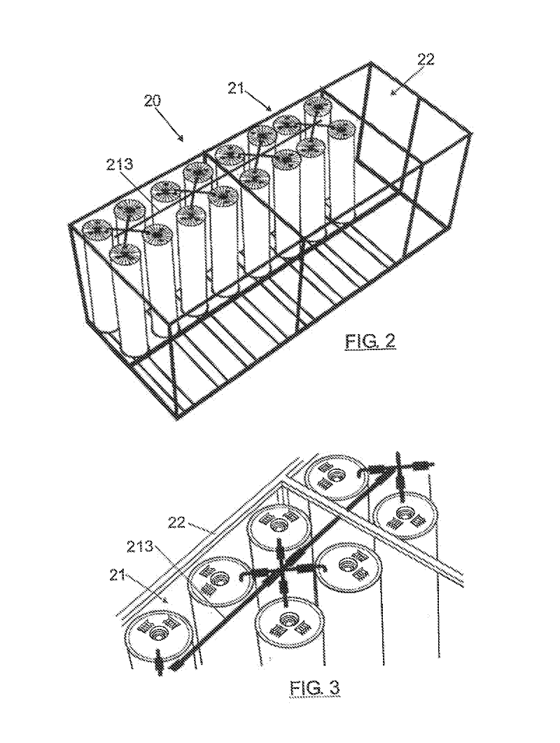

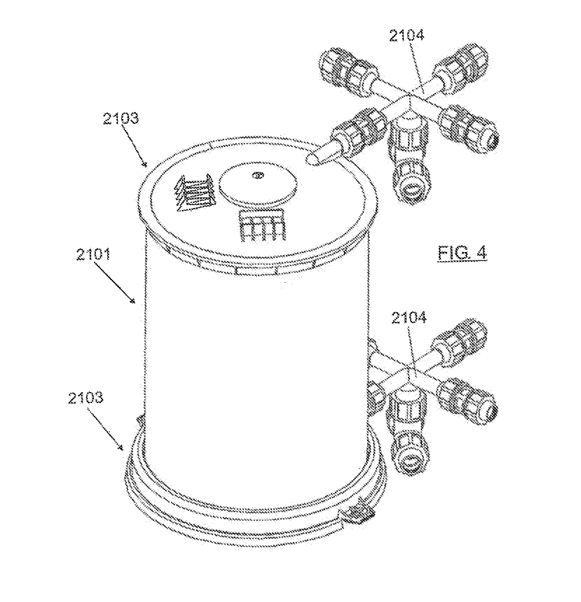

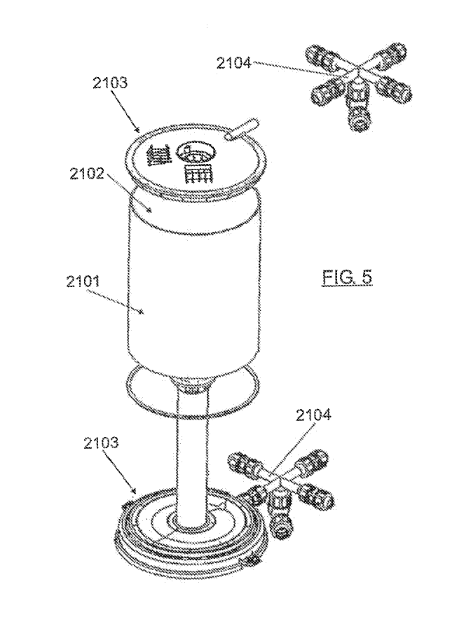

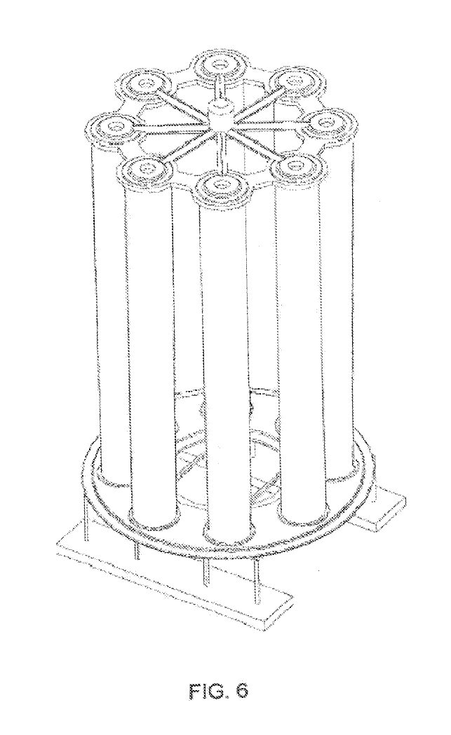

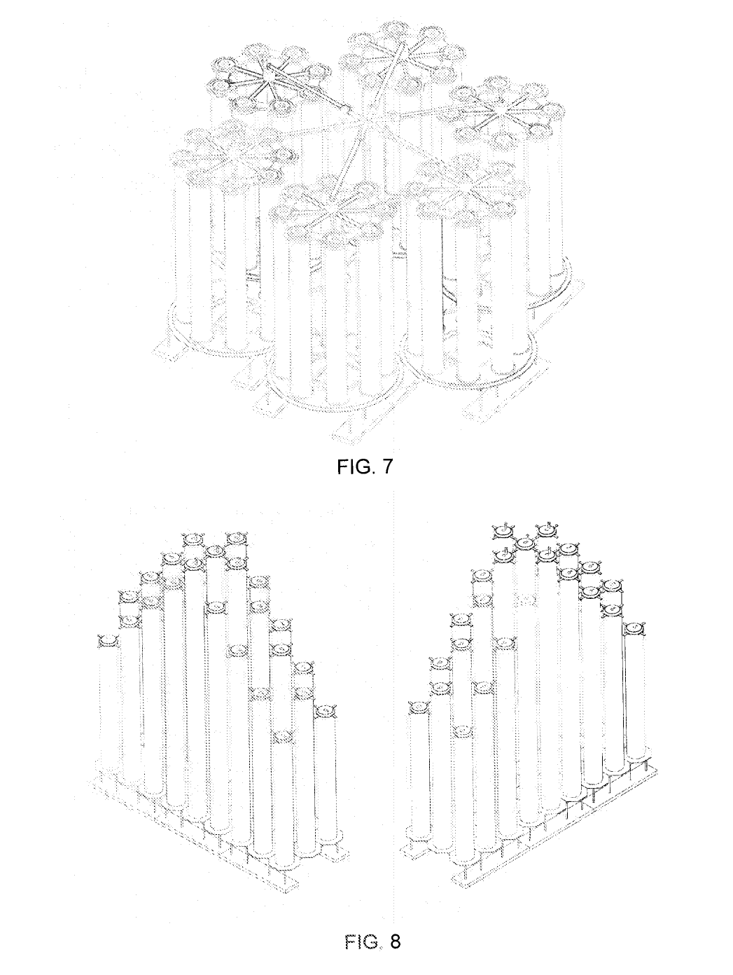

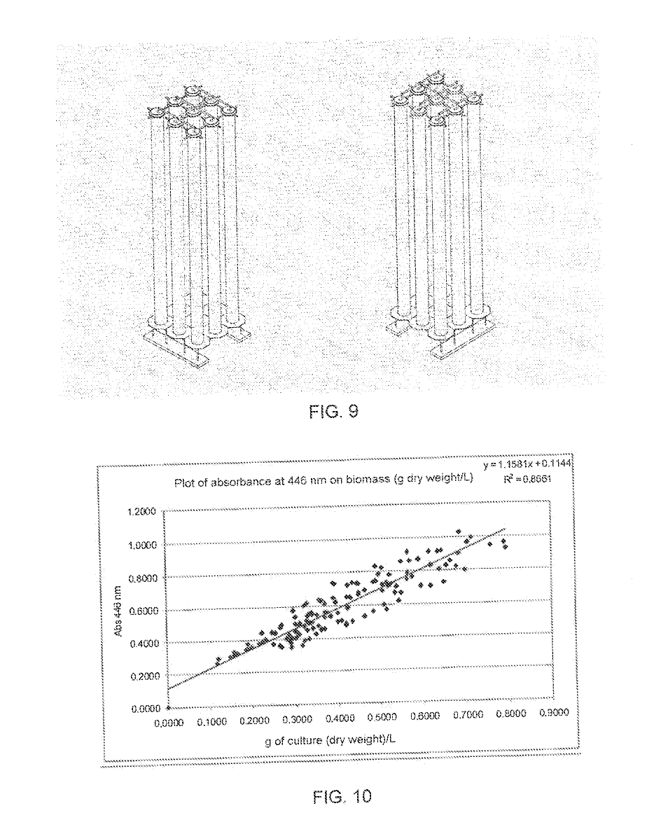

### PREFERRED EMBODIMENT OF THE INVENTION

As can be seen in the attached figures, the continuous system for the generation of high nutritional value and energy resources, in its preferred embodiment comprises:

(i) first production means (20) for producing phytoplankton culture (10), wherein said first means (20) in turn comprise:
(a) a plurality of production modules (21) for producing phytoplankton culture that are vertically placed, fed with CO₂ and interconnected to one another (213) in the form of a closed circuit, comprising a recirculation pump for recirculating the phytoplankton culture and a plurality of generation means for generating a magnetic field located in the recirculation pipes (213), such that either a continuous or alternating excitation magnetic field is generated in said area;
(b) a container structure (22) for containing the production modules, such that the modules-structure assembly is stackable and has dimensions such that its transport in a standard transport container is allowed;
(c) natural (sunlight, sunlight concentrators-distributors) and/or artificial (fluorescent tubes, LEDs) illumination means, wherein said illumination means can be internal or external with regard to the production modules for producing culture;

Working in parallel is thus achieved between the production modules (21), assuring the same amount of fluid in a margin comprised between 10% and 15% of flow variation and one and the same load for all the modules (21). The system also allows an amount of processing of the phytoplankton culture of up to 20000 liters/hour, preferably 5000 liters/hour.

The production modules (21) for producing phytoplankton culture (10) comprise a plurality of columns connected to one another, and wherein said columns in turn comprise:

(i) a tube assembly (210) with a maximum height of 10 meters and a preferred height between 2 and 2.25 meters containing the phytoplankton culture, said tube assembly (210) comprising at least one of the following:
(a) a first external tube (2101) comprising, in turn, at least one of the following layers:
   a first outer layer of ultraviolet treatment;
   a second intermediate layer of a material such that it is resistant to the pressure of the volume contained; and
   a third antiadherent inner layer, in contact with the contained fluid;
(b) a second internal tube (2102) with regard to the first tube (2101), said second tube comprising at least one of the following layers:
   a first antiadherent outer layer, in contact with the contained fluid;
   a second intermediate layer of a material resistant to the pressure of the volume contained in the first tube; and
   a third inner layer of ultraviolet treatment;
(c) upper and lower assembly closure elements (2103); and
(d) inter-column connection elements (2104), comprising a fluid connector between columns and a plurality of auxiliary fluid circulation elements;
(e) cleaning means comprising at least two concentric rings, a first outer ring and a second inner ring and wherein both are joined by means of a plurality of radii, and a peripheral element which cleans by friction the walls with which it comes into contact without damaging them;
(f) a cooling and/or refracting element (a third outer tube with regard to the first external tube which would act as a jacket), comprising in turn refracting subelements and/or a cooling and/or refracting liquid therein;
(g) a medium containing zeolites;

such that the second internal tube (2102) is inserted in the first external tube (2101), preferably concentrically, the assembly being closed at its ends, the inter-column connection elements (2104) being located both at its upper part and at its lower part; and

(ii) feed and turbulence generation means for generating turbulences of a fluid in gaseous state and for the feeding thereof, this fluid in gaseous state being at least one selected from:
(a) air;
(b) N₂;
(c) CO₂;
(d) industrial emission exhaust gases; or
(e) a combination thereof.

The production modules (21) for producing phytoplankton culture (10) comprise four production columns (210) for producing phytoplankton culture connected in parallel and/or in series.

The system likewise comprises second mechanical and/or thermal separation means (50) connected to the outlet of the first production means (20), such that the water present in the latter is eliminated after an emptying step (30).

The system comprises third transformation means (60) for transforming the dry product obtained in the second means (50), such that an energy resource (70) and/or value-added resources (90) and/or high nutritional value products (80) are obtained, wherein said third means comprise means selected from:
(a) gasification means, such that a gas suitable for its use as a fuel is obtained; or
(b) pyrolysis transformation means such that a product is obtained selected from:
   a fuel oil;
   a liquid mixture of organic compounds which, after a refining process, allows obtaining different fractions with physicochemical characteristics similar to those obtained from petroleum.
   an ester which, in combination with fatty acid dissolution and extraction means and esterification/transesterification means, allows obtaining a diesel compound;
(c) nutritional product extraction means;
(d) a combination of the above.

The second mechanical and/or thermal separation means comprise at least one of the following systems or means:
a submerged membrane filtration system (MBR);
a tangential filtration system;
flocculation and/or coagulation means;
electrocoagulation means;
an ultrasound system;
mechanical vapor recompression (MVR) means;
pressing means;
hot-air thermal drying means, such that concentrated product may be dried by means of hot air at a temperature of not less than 75° C.;
freeze-drying means;
multiple-effect evaporators;
decanting means; or
any combination of the above;
   such that up to 99.9% of the water present in the production means is eliminated.

In a practical embodiment, the source of the CO₂ used to generate turbulences and feed the phytoplankton culture is partial or complete feedback from the transformation and/or combustion means used for the resulting product.

In the same way, the system in another practical embodiment incorporates wind generators, such that said wind generators generate the actual electricity for the use of the system, an excess energy accumulation being generated in the phytoplankton culture itself.

Finally, in a last embodiment the system is installed, partially submerged, on floats, in the sea, lakes, rivers, or oceans.

And in another preferred embodiment of the invention, the production modules comprise means for generating an alternating and/or continuous excitation magnetic field located in the recirculation pipes (213), such that a magnetic field for excitation of the culture is generated there.

In still another preferred embodiment of the invention, the system includes solar panels and Fresnel mirrors.

And in yet another preferred embodiment of the invention, the system includes rotation means to maximize collection of solar radiation.

### Comparative Tests on the Effect of an Inner Tube on the Phytoplankton Cultures

a. Object of the Study

The object of the study was to compare the effect of differently designed bubble column/tube photobioreactors on the production of cultures of Tetraselmis sp.

The photobioreactors employed differed with respect to the following variables:
1. Photobioreactor type: All consisted of 2 m-tall vertical tubes, with some having an inner tube of varying diameter for illumination.
2. Photobioreactor material: Three transparent materials were tested, i.e., glass (VD), polycarbonate (PC) and methacrylate (MT).
3. Number of tubes making up the photobioreactor.
   b. Materials and Methods

### b.1. Culture Conditions and Production Data

The study was conducted at a pilot plant, taking care that all the photobioreactors were exposed to the same ambient photon flux density, 250 μmoles·m⁻²·s⁻¹, supplied by GROLUX lamps, particularly well-suited for plant growth, and to the same temperature, 20° C., by connecting all the photobioreactors to a heat exchanger.

Three experiments were carried out, with three replications each. Each replication lasted eight days. Replications were discontinuous, with each having the same initial biomass at the outset. The water used to start each replication was filtered sea water sterilized by ozonization using a PAP model COM AD08 ozone generator.

The amount of inoculum required for each replication to achieve the same cell concentration in all the photobioreactors was calculated using a calibration curve plotting biomass (g of culture expressed as dry weight/L of culture) on culture absorbance at 680 nm (FIG. 10). All replications started at an absorbance of 680 nm (uA=0.04), the equivalent of 0.23 g dry weight/L.

All the photobioreactors received a mixture of air and 2-% CO₂ previously sterilized by filtration (0.2 μm). The CO₂ supply was regulated by a timer according to the same cycle used for illumination, that is, the supply was halted during the darkness portion of the cycle.

Production data collection involved taking samples of the cultures considered at time 0 and every 24 h and determining the biomass production gravimetrically by vacuum filtration through Whatman GF/C filters and drying at 80° C. for 24 h. In addition to the determinations of biomass production, the pH, temperature, and dissolved oxygen level for all the cultures were continuously monitored.

### 2.2. Photobioreactor Design

Table 1 sets out the differentiating features (variables assessed) for the photobioreactors considered.

TABLE 1
Photobioreactor characteristics

| Sample | N1 | N2 | N6 | N9 |
|---|---|---|---|---|
| Material | PC | PC | MT | PC |
| φ Outer tube (mm) | 400 | 400 | 300 | 600 |
| φ Inner tube (mm) | 200 | 90 | 90 | 400 |
| Number of tube | 6 | 6 | 6 | 3 |
| Total volume (L) | 1060 | 1300 | 680 | 950 |

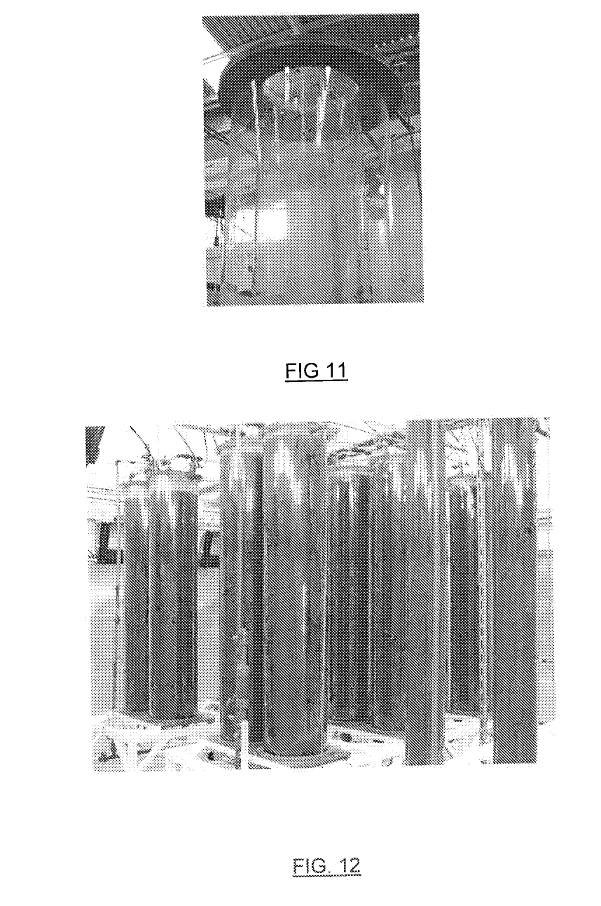

FIG. 12 depicts, from left to right, photobioreactors N1 and N2. FIG. 11 illustrates one of the columns in photobioreactor N9, depicting the inner tube.

### 2.3. Experimental Design

Experiment 1: Cultures with a photoperiod of 16:8 h (light:darkness). For the photobioreactors with an inner tube, there was a 70-W fluorescent bulb inside illuminated according to the same cycle of light:darkness.
Experiment 2: Cultures were grown with the same photoperiod as in the preceding experiment but without the fluorescent bulbs in the inner columns in the photobioreactors.
Three replications of all experiments were performed.

### 3. RESULTS AND CONCLUSIONS

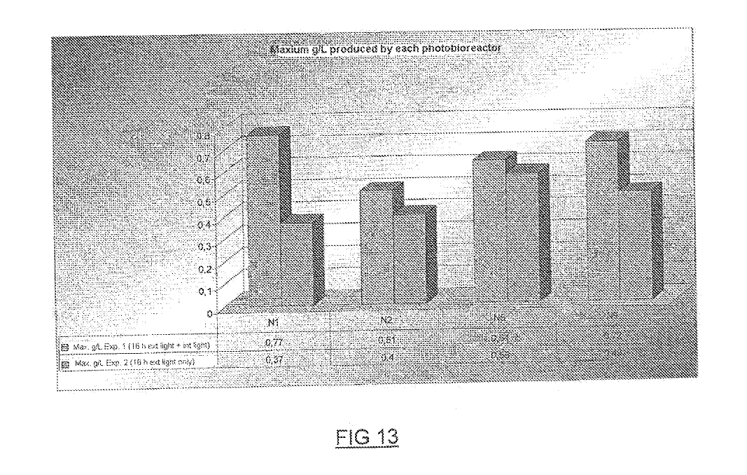

The mean values for the two experiments are summarized in Table 2 and depicted in FIG. 13.

TABLE 2
Mean final biomass outputs for the two experiments
Final biomass (g of culture (dry weight)/L)

| | N1 | N2 | N6 | N9 |
|---|---|---|---|---|
| Max. g/L Exp. 1 (16 h ext light + int light) | 0.77 | 0.51 | 0.64 | 0.72 |
| Max. g/L Exp. 2 (16 h ext light only) | 0.37 | 0.40 | 0.58 | 0.49 |

Thus, a quite substantial increase in final biomass output was observed in the photobioreactors in all cases in which an internal light source was used compared with the values obtained when an external light source was the sole light source used.

1. A system comprising:
(i) a first production unit for culturing at least one phytoplankton culture; and
(ii) an illuminating unit for naturally and/or artificially illuminating the phytoplankton culture to induce growth and/or multiplication of the culture,
wherein the said first production unit comprises at least one tube assembly which comprises the phytoplankton culture and the tube assembly comprises:
(a) a first outer tube that comprises the phytoplankton culture and allows exposure of the phytoplankton culture to at least one external light source, and
(b) a second tube inside the first outer tube that allows exposure of the phytoplankton culture to an internal light source that is independent of the external light source.

2. The system according to claim 1, wherein the first outer tube comprises at least one of the following layers:
a first, outer layer having an ultraviolet treatment;
a second, intermediate layer comprising a material resistant to the pressure of the phytoplankton culture that is in the first outer tube; and
a third, antiadherent, inner layer in contact with the phytoplankton culture.

3. The system according claim 1, wherein the second tube inside the first outer tube comprises at least one of the following layers:
a first, antiadherent, outer layer, in contact with the phytoplankton culture;
a second, intermediate layer comprising a material resistant to the pressure of the phytoplankton culture that is in the first outer tube; and
a third, inner layer having an ultraviolet treatment.

4. The system according to claim 1, wherein the second, inner tube is inserted concentrically within the first, outer tube and the assembly is closed at both ends of the first outer tube.

5. The system according to claim 1, wherein the tube assembly further comprises a third, cooling and/or refracting tube external to the first outer tube, such that it serves as a jacket around said first outer tube, and the third tube comprises at least one refracting component and/or a cooling and/or refracting fluid therein.

6. The system according to claim 1, wherein the tube assembly further comprises at least one cleaning unit comprising a first outer ring and a second inner ring fastened together and attached to a peripheral members which, by friction, cleans the walls with which they come into contact.

7. The system according to claim 6, wherein the first outer ring and the second inner ring of the cleaning unit are joined together by a plurality of radii.

8. The system according to claim 1, wherein the tube assembly further comprises top and bottom members for closing the assembly and a plurality of connecting columns for connecting auxiliary members for circulating fluids.

9. The system according to claim 1, further comprising at least one recirculation pipe, and wherein the tube assembly further comprises a magnetic field generating unit for generating a magnetic field located on the recirculation pipe to induce growth and/or multiplication of the culture.

10. The system according to claim 1, wherein the tube assembly further comprises a turbulence generating unit for generating turbulence in the phytoplankton culture by bubbling at least one gas selected from the group consisting of
(a) air;
(b) N₂;
(c) CO₂; and
(d) industrial emission exhaust gases.

11. The system according to claim 1, further comprising:
(iii) a second mechanical and/or thermal separation unit connected to an outlet of the first production unit; and
(iv) a third transformation unit for transforming the product produced by the second mechanical and/or thermal separation unit so that high nutritional value products and/or high value-added products and/or energy resources are obtained.

12. The system according to claim 11, wherein the second mechanical and/or thermal separation unit is at least one selected from the group consisting of:
a filtration unit;
a flocculation and/or a coagulation unit;
an electrocoagulation unit;
an ultrasonic system;
a mechanical vapor recompression (MVR) unit;
a centrifuging unit;
a pressing unit;
a thermal drying unit employing hot air and/or hot exhaust gases;
a freeze-drying unit;
multiple-effect evaporators; and
a decanting unit.

13. The system according to claim 11, wherein the third transformation unit is at least one selected from the group consisting of:
(a) a gasification unit, so that a gas suitable for use as fuel is obtained;
(b) a thermochemical pyrolytic transformation unit operating at all pressure ranges; and
(d) a nutritional product extraction unit.

14. The system according to claim 1, further comprising at least one of the following unit:
(v) a structural unit for holding first production unit;
(vi) a concentration unit for concentrating solar radiation;
(vii) a rotation unit to maximize collection of the solar radiation; and
(viii) a structure for holding production modules whereby a module-structure assembly of the production modules is stackable and in a size to allow transportation in a standard transport container.

15. The system according to claim 14, wherein the concentration unit for concentrating solar radiation is selected from the group consisting of solar panels, Fresnel mirrors, light collecting and concentrating systems, solar radiation condensers, and light wells made of reflecting material.

16. The system according to claim 11, wherein CO₂ fed into the phytoplankton culture in the first production unit comes in part or in whole from partial or complete feedback of the surplus CO₂ from the transformation unit.

17. The system according to claim 1 wherein the system is installed, partially submerged, on floats, in the sea, lakes, rivers, or oceans.

18. The system according to claim 1, wherein the external light source is at least one external light source selected from the group consisting of natural light, either direct or concentrated by solar concentrators, and artificial light comprising light from fluorescent lamps, incandescent lamps, LEDs, halogen lamps, and light generation sources by compounds excitable by radioactivity.

19. The system according to claim 1, wherein the internal light source is at least one internal light source selected from the group consisting of natural light, either direct or concentrated by solar concentrators, and artificial light comprising light from fluorescent lamps, incandescent lamps, LEDs, halogen lamps, and light generation sources comprising compounds excitable by radioactivity.
